(19) United States
(12) Patent Application Publication
(10) Pub. No.: US 2025/0037865 A1
(43) Pub. Date: Jan. 30, 2025

(54) MEDICAL DATA PROCESSING

(71) Applicant: KONINKLIJKE PHILIPS N.V., EINDHOVEN (NL)

(72) Inventors: Wei NI, SHANGHAI (CN); En OUYANG, SHANGHAI (CN); Ken CHEN, SHANGHAI (CN); Binyang HU, SHANGHAI (CN); Liang TAO, SHANGHAI (CN)

(21) Appl. No.: 18/716,971

(22) PCT Filed: Nov. 25, 2022
(86) PCT No.: PCT/EP2022/083360

§ 371 Date: Jun. 6, 2024

(30) Foreign Application Priority Data
Dec. 9, 2021 (WO) PCT/CN2021/136896
Apr. 7, 2022 (EP) 22167053.2

(51) Int. Cl. G16H 50/20; G16H 10/60

(52) U.S. Cl. CPC G16H 50/20; G16H 10/60

(57) ABSTRACT

According to embodiments of the present disclosure, there is provided a solution for medical data processing. In this solution, a first set of entities and a second set of entities are respectively extracted from a first medical report and a second medical report related to a patient. A semantic correlation is determined between the first set of entities and the second set of entities. A concordance level between medical information in the first medical report and medical information of the second medical report is determined based on the semantic correlation. In this way, the cross-report concordance analysis can be automatically conducted based on semantic correlation of entities extracted from the reports.

### FIELD

Embodiments of the present disclosure relates generally to data processing, and more specifically, to method, computing system and computer readable medium for medical data processing.

### BACKGROUND

In the field of medical data processing, various medical reports, such as radiology reports, pathology reports, medical laboratory reports, ultrasound reports and discharge summary reports, may be used in diagnostic, treatment, surgery, and other medical procedures for patients. For example, in a diagnostic procedure, physicians usually refer to information in different medical reports of a patient to derive a medical diagnosis. Analyzing information in different medical reports may involve cross-report correlation analysis, which is useful for peer learning, continuing education, quality assurance, specimen adequacy, regulatory requirements, and the like.

Cross-report correlation is to analyze the cross-report concordance and ensure that there are adequate, accurate and representative radiograph samples, so as to enhance quality of patient care, help meet regulatory requirements, support multidisciplinary conferences, and also facilitate peer learning and continuing education. Nowadays, cross-report correlation analysis has been conducted manually. Such large-scale manual correlation analysis is tedious and time-consuming, and is hence not feasible for clinical practices. Therefore, it is desirable for a more intelligent system to better perform cross-report correlation analysis.

### SUMMARY

According to embodiments of the present disclosure, there is provided a solution for cross-report correlation analysis.

In a first aspect, there is provided a method for data processing. The method comprises extracting a first set of entities from a first medical report. The first medical report presents first medical information related to a patient. The method further comprises extracting a second set of entities from a second medical report. The second medical report presents second medical information related to the patient. The method further comprises determining a first semantic correlation between the first set of entities and the second set of entities; and determining, based on the first semantic correlation, a first concordance level between the first medical information in the first medical report and the second medical information in the second medical report.

In a second aspect, there is provided a computing system. The computing system comprises: at least one processor; and at least one memory comprising computer readable instructions. The instructions when executed by the at least one processor of an electronic device, cause the electronic device to perform operations comprising: extracting a first set of entities from a first medical report, the first medical report presenting first medical information related to a patient; extracting a second set of entities from a second medical report, the second medical report presenting second medical information related to the patient; determining a first semantic correlation between the first set of entities and the second set of entities; and determining, based on the first semantic correlation, a first concordance level between the first medical information in the first medical report and the second medical information in the second medical report.

In a third aspect, there is provided a computer readable medium comprising instructions which, when executed by at least one processor, cause the at least one processor to perform one or more embodiments of the method of the first aspect.

The Summary is to introduce a selection of concepts in a simplified form that are further described below in the Detailed Description. The Summary is not intended to identify key features or essential features of the present disclosure, nor is it intended to be used to limit the scope of the present disclosure.

### BRIEF DESCRIPTION OF THE DRAWINGS

Through the following detailed descriptions with reference to the accompanying drawings, the above and other objectives, features and advantages of the example embodiments disclosed herein will become more comprehensible. In the drawings, several example embodiments disclosed herein will be illustrated in an example and in a non-limiting manner, where:

FIG. 1 illustrates an example environment in which various embodiments for medical data processing in accordance with the present disclosure can be implemented;

FIG. 2 illustrates a block diagram of an example subsystem for medical report selection in accordance with some embodiments of the present disclosure;

FIG. 3 illustrates an example block diagram of example architecture for determining concordance level between reports in accordance with some embodiments of the present disclosure;

FIG. 4 illustrates an example block diagram of example architecture for determining concordance level between subsets of entities in accordance with some embodiments of the present disclosure;

FIG. 5 illustrates an example user interface in accordance with some embodiments of the present disclosure;

FIG. 6 illustrates another example user interface in accordance with some embodiments of the present disclosure;

FIG. 7 illustrates a flowchart of a method for data processing in accordance with an implementation of the present disclosure; and

FIG. 8 illustrates a simplified block diagram of a device that is suitable for implementing embodiments of the present disclosure.

Throughout the drawings, the same or similar reference symbols refer to the same or similar elements.

### DETAILED DESCRIPTION OF EMBODIMENTS

Principles of the present disclosure will now be described with reference to some example embodiments. It is to be understood that these embodiments are described only for the purpose of illustration and help those skilled in the art to better understand and thus implement the present disclosure, without suggesting any limitations to the scope of the subject matter disclosed herein.

As used herein, the term “includes” and its variants are to be read as open terms that mean “includes, but is not limited to.” The term “based on” is to be read as “based at least in part on.” The terms “an implementation” and “one implementation” are to be read as “at least one implementation.” The term “another implementation” is to be read as “at least one other implementation.” The term “first,” “second,” and the like may refer to different or the same objects. Other definitions, either explicit or implicit, may be included below.

As used herein, a “machine learning model” is an AI model, which may also be referred to as a “learning model”, “learning network”, “network model”, or “model.” These terms are used interchangeably hereinafter. A deep learning model is one example machine learning model, examples of which include a “neural network.” A parameter set of the machine learning model is determined through a training phrase of the model based on training data. The trained machine learning model maps a received input to a corresponding output using the trained parameter set. Therefore, the training process of a machine learning model may be considered as learning, from the training data, a mapping or association between the input and the output.

As briefly mentioned above, analyzing information in different medical reports such as cross-report correlation analysis is of great importance in medical area. For example, in image-guided breast biopsies, a lesion is usually detected at the end of the radiology procedure(s). The radiology interpretation provides the imaging characteristics of this lesion. These imaging characteristics may then be correlated to the histopathological diagnosis. If the imaging characteristics of the lesion correspond to the pathological diagnosis, e.g., when if suspicious imaging findings and malignant pathology diagnoses are found, the correlation result is deemed concordant. On the other hand, if the imaging characteristics are found to be different from the pathological diagnosis, e.g., if both suspicious imaging findings and benign pathology diagnoses are detected, the correlation result is deemed discordant. Such confirmation of radiology-pathology concordance also helps ensure appropriate patient care.

However, nowadays, the cross-report correlation analysis is usually conducted manually. Such manual cross-report correlation analysis is tedious and time-consuming, especially for large-scale practices. Therefore, it is desirable for a more intelligent system to better perform cross-report correlation analysis.

It has been proposed to perform a rule based automatic cross-report correlation by making each biopsy-pathology correlation as concordant or discordant. However, such approach is lack of comprehensive coverage across several different report types, such as radiology examinations, biopsies, and pathology reports. In addition, such rule based approach usually utilizes a rule based model, which is difficult to be generalized and tailored for new application scenarios. It is also difficult to fine-tune the rule based algorithm from institution to institution based on individual preference.

It has also been proposed to conduct an automated cross-report correlation procedure by setting a cutoff value to limit the correlation across different report within a specific time period. However, such approach limits the coverage within the specific time period. In addition, such approach is also lack of comprehensive coverage across several different reports types.

In view of the above, it is desirable for a more intelligent system which can conduct automated cross-report correlation between various types of reports and during different timespan of reports. According to embodiments of the present disclosure, there is proposed an improved solution for cross-report correlation. In this solution, a first set of entities and a second set of entities are extracted from a first medical report and a second medical report, respectively. The first and second sets of entities respectively present first medical information and second medical information related to a patient. A concordance level between the first medical information in the first medical report and the second medical information in the second medical report will be determined based on a semantic correlation between the first set of entities and the second set of entities.

By determining the concordance level between different reports based on the semantic correlation between entities extracted from the different reports, the concordance result may be more meaningful. Moreover, such semantic based approach can be adapted to various types of medical reports taking at different time. The coverage of types of reports and the coverage of timespan of reports will be broadened. In this way, it may provide a more generalizable and more robust cross-report correlation analysis for various institutional and individual needs.

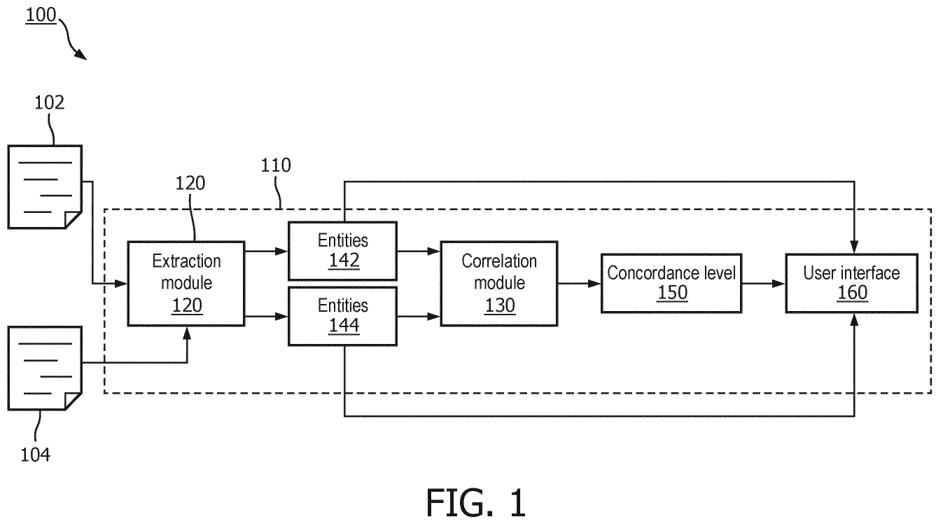

Example embodiments of the present disclosure will be discussed in detail below with reference to FIGS. 1-8. FIG. 1 illustrates an example environment 100 in which various embodiments for medical data processing in accordance with the present disclosure can be implemented. It is to be understood that the environment 100 shown in FIG. 1 is only for the purpose of illustration, without suggesting any limitation to functions and the scope of the embodiments of the present disclosure.

In the environment 100, a data processing system 110 is configured to perform various processes relating to medical reports. For example, the data processing system 110 may perform cross-report correlation analysis between a medical report 102 and a medical report 104. As used herein, the term of “medical document” or the “medical report” may be a radiology report, a pathology report, a medical laboratory report, an ultrasound report and a discharge summary report, etc. In some example embodiments, the medical report is a digital medical report. Alternatively, the medical report may be a paper medical report which is inputted to the data processing system 110 may converted to a corresponding digital medical report by the data processing system.

Each patient may have a plurality of medical reports. To conduct a cross-report correlation analysis for a particular patient, each time the data processing system 110 may select two medical reports of the particular patient for analysis. The data processing system 110 may select two medical reports of the particular patient by various ways. In some example embodiments, the data processing system 110 may select two latest medical reports of different types for cross-report correlation analysis. Alternatively, the data processing system 110 may randomly select two medical reports of different types for cross-report correlation analysis. For example, the data processing system 110 may randomly select an ultrasound report and a pathology report for cross-report correlation analysis.

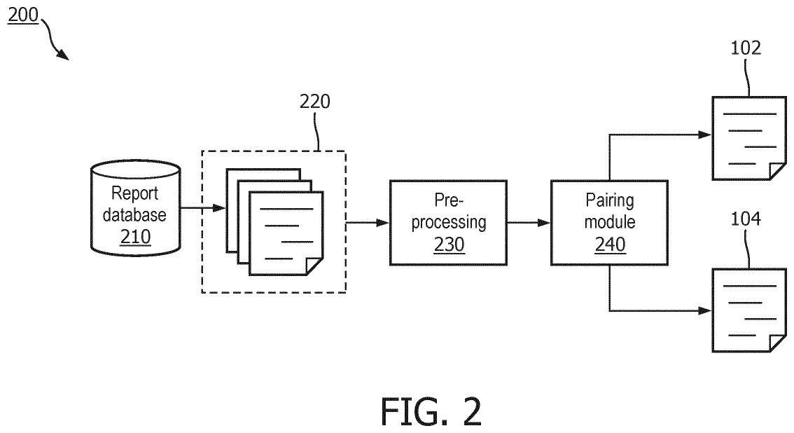

FIG. 2 illustrates a block diagram of an example subsystem 200 for medical report selection in accordance with some embodiments of the present disclosure. It is to be understood that the subsystem 200 shown in FIG. 2 is only for the purpose of illustration, without suggesting any limitation to functions and the scope of the embodiments of the present disclosure. The subsystem 200 may be included in the data processing system 110. It is to be understood that the subsystem 200 may also be implemented at a separate device communicating with the data processing system 110. For the purpose of discussion, the subsystem 200 will be described with reference to FIG. 1.

As shown in FIG. 2, the subsystem 200 includes a report database 210 which is configured to store various types of medical reports for different patients. For example, the radiology, biopsy and pathology and other medical reports may be extracted from Health Level 7 result messages and stored in the report database 210 along with pertinent patient and examination metadata. Each medical report may be stored as raw data in free text in the report database 210. The report database 210 may be a MySQL database, or Oracle database or other suitable database.

To perform cross-report correlation analysis for a patient, the subsystem 200 may retrieve a plurality of medical reports 220 for the patient from the report database 210. In some example embodiments, the subsystem 200 may perform a pre-processing 230 for the plurality of medical reports 220. For example, the pre-processing 230 may comprise removing the punctuations, numbers, and stop words from the raw data in each of the plurality of medical reports 220.

In addition, in some example embodiments, the pre-processing 230 may comprise additional procedure(s). For example, in the scenario that the medical reports are in for example Chinese language, the pre-processing 230 may comprising an additional word segmentation procedure to transform entire texts in the medical report(s) into a sequence of words. It is to be understood that the above described Chinese language and example pre-processing procedure is only for the purpose of illustration, without suggesting any limitations. The medical reports may be in any suitable language. The pre-processing 230 may comprise any suitable processing procedure.

As shown in FIG. 2, the subsystem 200 may further comprise a pairing module 240. The pairing module may be configured to select the medical report 102 and the medical report 104 from the plurality of medical reports 220 or optional the pre-processed plurality of medical reports 220. The medical report 102 and the medical report 104 may be of different report types. For example, the medical report 102 may be a radiology report, while the medical report 104 may be a pathology report.

In some example embodiments, the pairing module 240 may determine text similarities between the plurality of medical reports 220 of the patient. The pairing module 240 may select the medical reports 102 and 104 from the plurality of medical reports 220 based on the text similarities.

In some example embodiments, the pairing module 240 may comprise a pre-trained language model. For example, the pairing module 240 may comprise a Doc2Vec model, which is an extension to Word2Vec model. The Doc2Vec model may be a shallow, two-layer neural networks which is trained to reconstruct linguistic contexts of reports or documents. The Word2Vec model takes as its input a large corpus of words and produces a vector space of multiple dimensions, with each unique word in the corpus being assigned with a corresponding vector in the space. Likewise, the Doc2Vec model may further produce a document vector together with the word vectors.

With the Doc2Vec model, the pairing module 240 may determine a document vector for each of the plurality of medical reports. The pairing module 240 may apply cosine similarity or Jaccard similarity to the document vectors to evaluate the text similarities between the plurality of medical reports 220. It is to be understood that the Doc2Vec model is only for the purpose of illustration. Any suitable neural network model can be used to implement the pairing module 240.

In some example embodiments, the medical report 102 and the medical report 104 may be two medical reports with a top cosine similarity. Alternatively, the pairing module 240 may initially select for example a latest radiology report as the medical report 102. The pairing module 240 may select a medical report with a top similarity with the medical report 102 as the medical report 104.

By selecting two medical reports based on the text similarities by the subsystem 200, the data processing system 110 may automatically and adaptively select the best report pair from the plurality of medical reports. For example, when the doctor intends to take the radiology report into consideration, and wants to find another type of report to conduct cross-report correlation analysis for better results, the present data processing system may select a more suitable medical report to be analyzed with the radiology report. In this way, it may reduce the time and computation cost of the data processing procedure.

Example embodiments regarding selecting two medical reports in accordance with some embodiments of the present disclosure have been described with respect to FIG. 2. It is to be understood that although there are only two medical reports to be selected to conduct cross-report correlation analysis as shown in FIGS. 1-2, the data processing system 110 may select more than two medical reports to conduct cross-report correlation analysis.

Referring back to FIG. 1, the data processing system 110 may further comprise an extraction module 120. The extraction module 120 is configured to extract a set of entities 142 from the medical report 102, and extract a set of entities 144 from the medical report 104. As used herein, the term “entity” may be an embedded vector or other suitable data structure which represents a word or phrase associated with a medical concept, a relation or other medical term. Examples of medical concept represented by the “entities” may comprise but not limited to “tumor”, “left lung”, “boundary”, “shadow”, etc. As used herein, extracting entities from the medical report may also be referred to as embedding the medical report as entities.

In some example embodiments, the extraction module 120 may extract the set of entities 142/144 from the medical report 102/104 by using entity embedding algorithms. The entity embedding algorithms may be based on a knowledge base with adequate entities and relations. For example, the entity embedding algorithms may be based on a medical ontology knowledge base. Examples of knowledge bases may comprise but not limited to MeSH (Medical Subject Headings) or CMeSH (Chinese Medical Subject Headings). For example, the CMesh contains 391,892 medical concepts and 2,047,749 relations. It is to be understood that the MeSH and CMeSH are only for the purpose of illustration, without suggesting any limitation.

As illustrated in FIG. 1, the data processing system 110 further comprises a correlation module 130. The correlation module 130 is configured to determine a semantic correlation between the set of entities 142 and the set of entities 144. For example, the extraction module 120 may transmit the sets of entities 142 and 144 to the correlation module 130. In addition, the extraction module 120 may concatenate the set of entities 142/144 into an input matrix.

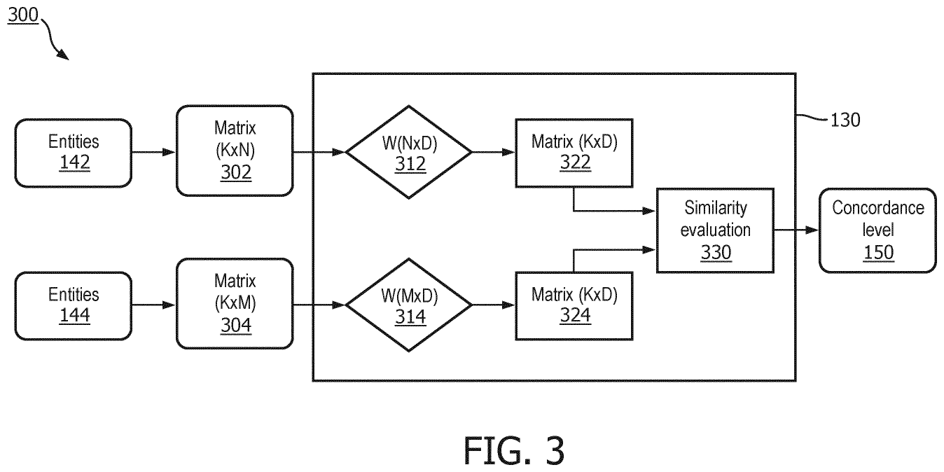

It is to be understood that different medical reports may comprise same or different numbers of entities. For example, the number of entities in the set of entities 142 may be different from the number of entities in the set of entities 144. Accordingly, the first input matrix concatenated by the set of entities 142 may be of different dimension from the second input matrix concatenated by the set of entities 144. The correlation module 130 may determine the semantic correlation between the first input matrix and the second input matrix by computing non-linear correlations. For example, the correlation module 130 may use the canonical-correlation analysis (CCA), the kernel principal component analysis (kernel PCA) or the kernel canonical-correlation analysis (kernel CCA) to determine the semantic correlation. Details regarding determining the semantic correlation will be described below with respect to FIG. 3.

As illustrated in FIG. 1, the correlation module 130 may determine a concordance level 150 between medical information in the medical report 102 and medical information in the medical report 104 based on the semantic correlation. The concordance level 150 may represent whether the medical report 102 matches or partially matches with the medical report 104.

FIG. 3 illustrates an example block diagram of example architecture 300 for determining concordance level between medical reports in accordance with some embodiments of the present disclosure. It is to be understood that the architecture 300 as shown in FIG. 3 is only for the purpose of illustration, without suggesting any limitation to functions and the scope of the embodiments of the present disclosure. The concordance level determination shown in the architecture 300 may be performed by the data processing system 110 in the FIG. 1 or any other suitable device. For the purpose of discussion, the architecture 300 will be described with reference to FIG. 1

As shown in FIG. 3, the set of entities 142/144 may be concatenated into a matrix 302/304. Each entity in the sets of entities 142 and 144 may be an embedded vector of K dimension. The medical report 102 may contain N entities, while the medical report 104 may contain M entities. Accordingly, the matrix 302 may be of dimension K×N, while the matrix 304 may be of dimension K×M. It is to be understood that K, N and M may be any suitable numbers. The number K may be preconfigured by the extraction module 120. The numbers N and M may be determined by the content of the medical reports 102 and 104, respectively.

In the example of FIG. 3, the correlation module 130 may multiply a cross-covariance matrix W 312 with dimension N×D to the matrix 302, to obtain a resultant product matrix 322 which is of dimension K×D. Likewise, the correlation module 130 may multiply a cross-covariance matrix W 314 with dimension M×D to the matrix 304, to obtain a resultant product matrix 324 which is of dimension K×D. The number D may be pre-configured by the correlation module 130.

By multiplying the cross-covariance matrices, the correlation module 130 may transform the matrices 302 and 304 into matrices with the same dimension. The correlation module 130 may comprise a similarity evaluation component 330. In some example embodiments, the similarity evaluation component 330 may use similarity evaluation metrics such as cosine similarity or Jaccard similarity or other suitable similarity metrics to determine the semantic correlation between the set of entities 142 and the set of entities 144. For example, the similarity evaluation component 330 may apply the cosine similarity to the matrix 332 and the matrix 334 to determine the semantic correlation.

The semantic correlation between the matrix 322 and the matrix 324 may represent the semantic correlation between the medical information in the medical report 102 and the medical information in the medical report 104. As used herein, the medical information contained in the medical report 102 may be referred to as the first medical information. Likewise, the medical information contained in the medical report 104 may be referred to as the second medical information. The semantic correlation between the matrix 322 and the matrix 324 may represent the correlation between the first and second medical information.

The similarity evaluation component 330 further determines the concordance level 150 based on the semantic correlation. For example, the similarity evaluation component 330 may perform a linear mapping between the semantic correlation and the concordance level 150. For example, a higher semantic correlation may be mapped to a higher concordance level 150, for example a value close to and less than 1. A lower semantic correlation may be mapped to a lower concordance level 150, for example a value close to and greater than 0. It is to be understood that the values of 0 and 1 are only for the purpose of illustration, without suggesting any limitations. The value of concordance level 150 may be of any suitable range.

Several embodiments regarding determining the concordance level have been described with respect to FIG. 3. It is to be understood that the architecture 300 in FIG. 3 is only for the purpose of illustration. It can also use other suitable process to determine the concordance level.

To this end, the data processing system 110 can determine the concordance level by determining the semantic correlation between entities extracted from different reports. In this way, such cross-report analysis based on the semantic meanings of different reports will provide more meaningful results comparing to the rule based approaches. Moreover, such semantic based approach is not limited to specific types of reports or specific timespan of reports. That is, the present semantic based cross-report analysis can be applied to various types of reports and whole timespan of different reports. Moreover, by using the correlation module, the present semantic approach can be generalizable and tailorable to institutional and individual needs, and the correlation result could be more robust.

Still referring to FIG. 1, the data processing system 110 may pre-determine a threshold level. If the concordance level 150 exceeds the threshold level, then the data processing system 110 may determine that the medical report 102 matches with the medical report 104. That is, the medical information of the medical report 102 matches with the medical information of the medical report 104. If the concordance level 150 is less than the threshold level, then the data processing system 110 may determine that the medical report 102 mismatches with the medical report 104.

Alternatively, or in addition, the data processing system 110 may predetermine a first threshold level and a second threshold level larger than the first threshold level. If the concordance level 150 is less than the first threshold level, then the data processing system 110 may determine that the medical report 102 mismatches with the medical report 104. That is, the medical information of the medical report 102 mismatches with the medical information of the medical report 104. If the concordance level 150 exceeds the first threshold level but is less than the second threshold level, the data processing system 110 may determine that the medical report 102 partially matches with the medical report 104. That is, the medical information of the medical report 102 partially matches with the medical information of the medical report 104. If the concordance level 150 exceeds the second threshold level, then the data processing system 110 may determine that the medical report 102 matches with the medical report 104. That is, the medical information of the medical report 102 matches with the medical information of the medical report 104.

By determining the concordance level 150, the data processing system 110 can provide information regarding the concordance between different medical reports. For example, the concordance level will indicate that two medical reports matches, partially matches or mismatches with each other. In some embodiments, the data processing system 110 may provide information regarding the concordance level between different medical reports via a user interface 160 as shown in FIG. 1. The user interface may receive the concordance level 150 and provide an indication based on the concordance level 150.

For example, if the concordance level 150 is less than the first threshold level, then the user interface 160 may provide an indication indicating that the medical information of the medical report 102 matches with the medical information of the medical report 104. If the concordance level 150 exceeds the first threshold level and is less than the second threshold level, then the user interface 160 may provide an indication indicating that the medical information of the medical report 102 partially matches with the medical information of the medical report 104. If the concordance level 150 exceeds the second threshold level, then the user interface 160 may provide an indication indicating that the medical information of the medical report 102 mismatches with the medical information of the medical report 104.

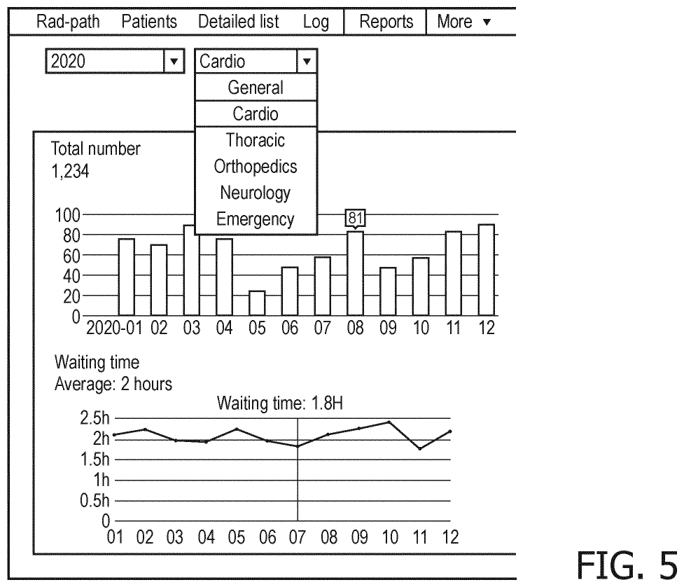

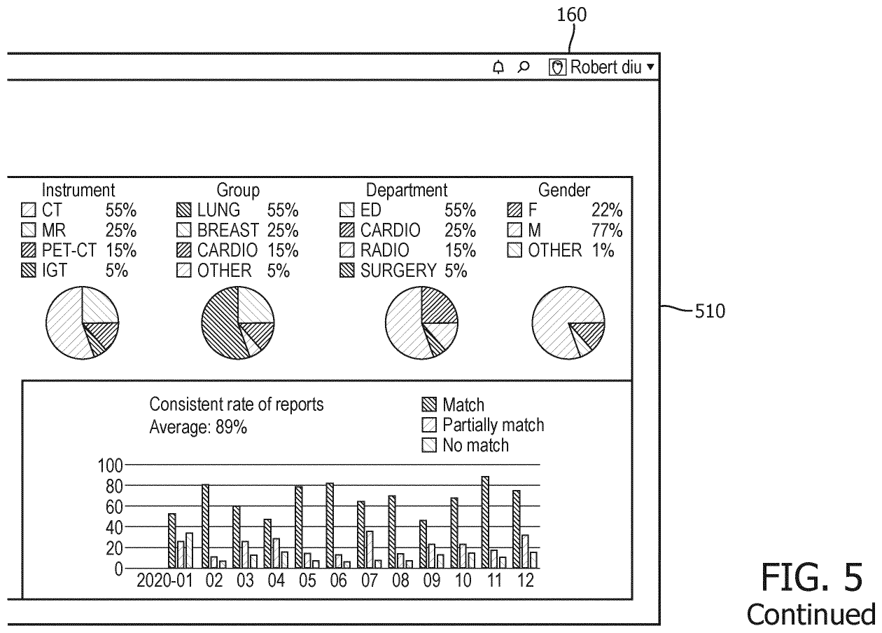

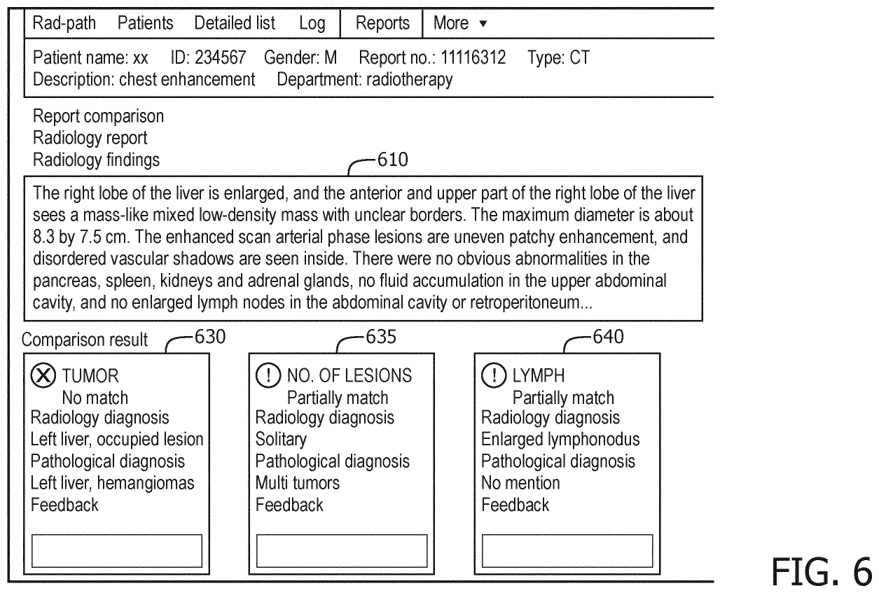

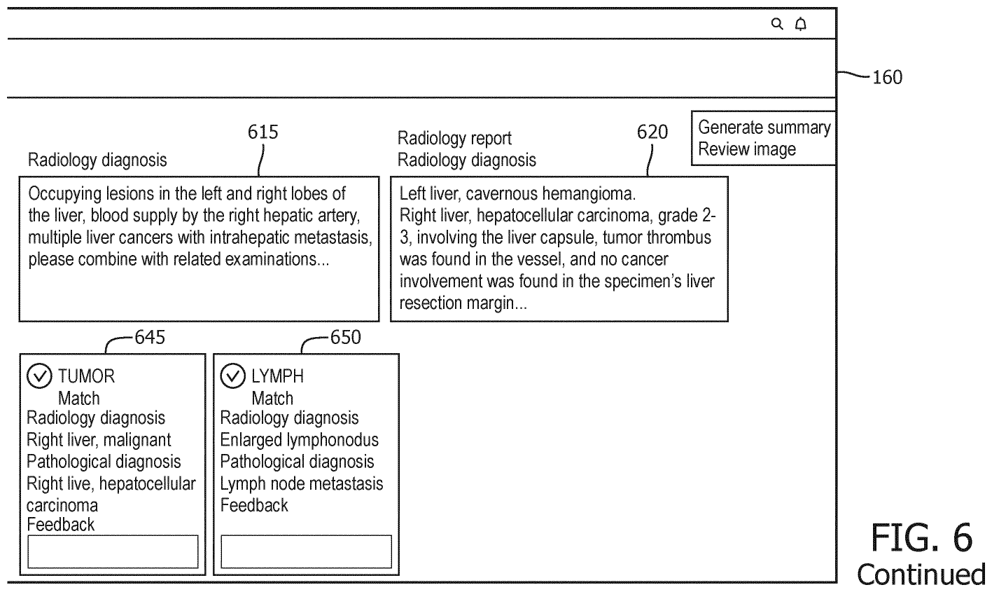

Likewise, in the examples that the data processing system 110 predetermines one threshold level, the user interface 160 may provide an indication indicating the match or the mismatch (no match) based on the concordance level 150 and the threshold level. It is to be understood that the above mentioned threshold level, the first and second threshold level could be predetermined as any suitable values, such as values between 0 and 1. The user interface 160 may display additional information other than the indication. Examples of display of the user interface 160 may be described with respect to FIGS. 5-6 below.

By providing the indication by the user interface 160, it may help the users of the data processing system 110, such as doctors, radiologists and other healthcare professionals, to obtain comparison information between different medical reports. In this way, it may help the users to make diagnosis, and also facilitate peer learning and continuing education.

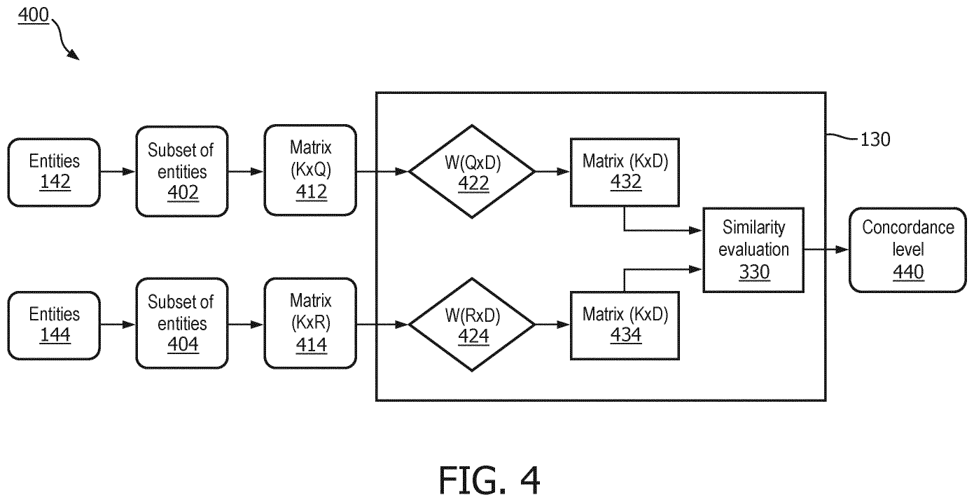

Several embodiments regarding determining the concordance level between medical reports have been described with respect to FIGS. 1 and 3. In some example embodiments, the data processing system 110 may further determine one or more concordance levels between subset(s) of entities from the medical report 102 and subset(s) of entities from the medical report 104. FIG. 4 illustrates an example block diagram of example architecture 400 for determining concordance level between subsets of entities in accordance with some embodiments of the present disclosure. It is to be understood that the architecture 400 shown in FIG. 4 is only for the purpose of illustration, without suggesting any limitation to functions and the scope of the embodiments of the present disclosure. The concordance level determination shown in the architecture 400 may be performed by the data processing system 110 in the FIG. 1. For the purpose of discussion, the architecture 400 will be described with reference to FIG. 1.

In some example embodiments, the data processing system 110 may associate each entity of the set of entities 142 and the set of entities 144 with a respective symptom of a disease or a respective body part. For example, the data processing system 110 may perform such association based on a knowledge base, such as a medical ontology knowledge base. The knowledge base comprises a plurality of medical concepts and relations. Examples of knowledge bases may comprise but not limited to MeSH or CMeSH.

As shown in FIG. 4, the data processing system 110 may determine a subset of entities 402 from the set of entities 142 and a subset of entities 404 from the set of entities 144. The subset of entities 402 and the subset of entities 404 are associated with a same target symptom of a disease or a same target body part. The subset of entities 402 may comprise medical information associated with the target symptom of a disease or the target body part in the medical report 102, while the subset of entities 404 may comprise medical information associated with the same target symptom of a disease or the same body part in the medical report 104.

For example, if the doctor is interested in the liver of the patient, the data processing system 110 may determine the subset of entities 402/404 associated with the liver from the set of entities 142/144. Examples of entities comprised in the subset of entities 402/404 may comprise embedded vectors representing “right liver”, “left liver”, “right hepatic”, “intrahepatic metastasis”, etc. For another example, if the doctor in interested in the lymphoma, the data processing system 110 may determine the subset of entities 402/404 associated with the lymphoma from the set of entities 142/144.

In some example embodiments, the data processing system 110 may concatenate the subset of entities 402/404 into a corresponding matrix 412/414. As aforementioned, each entity may be of dimension K. In the example shown in FIG. 4, the subset of entities 402 comprises Q entities, while the subset of entities 404 comprises R entities. Accordingly, the matrix 412 is of dimension K×Q, while the matrix 414 is of dimension K×R.

Similar to the concordance level determination process described with respect to FIG. 3, the data processing system 110 may determine a concordance level 440 between the subset of entities 402 and the subset of entities 404 by using the correlation module 130. For example, the correlation module 130 may multiply a cross-covariance matrix W 422 with dimension Q×D to the matrix 412, to obtain a resultant product matrix 432 which is of dimension K×D. Likewise, the correlation module 130 may multiply a cross-covariance matrix W 424 with dimension R×D to the matrix 414, to obtain a resultant product matrix 434 which is of dimension K×D.

By multiplying different cross-covariance matrix, the correlation module may transform the subsets of entities 402 and 404 into matrices 432 and 434 with the same dimension. In this way, the semantic correlation and the concordance level between medical information in different subsets may be determined. Although the matrices 432 and 434 shown in FIG. 4 are of the same dimension with the matrices 322 and 322 shown in FIG. 3, it is to be understood that the matrices 432 and 434 may be of different dimension with the matrices 322 and 322.

In some example embodiments, the data processing system 110 may use a single correlation module 130 to determine the concordance level between medical reports as well as the concordance level between different subsets of entities. Alternatively, the data processing system 110 may use two separate correlation modules to determine different concordance levels.

In some example embodiments, the similarity evaluation component 330 comprised in the correlation module 130 may use similarity evaluation metrics such as cosine similarity or Jaccard similarity or other suitable similarity metrics to determine a semantic correlation between the subset of entities 402 and the subset of entities 404. For example, the similarity evaluation component 330 may apply the cosine similarity to the matrix 432 and the matrix 434 to determine the semantic correlation.

The semantic correlation may represent the correlation between the medical information contained in the subset of entities 402 and the medical information contained in the subset of entities 404 in the medical report 104. As used herein, the medical information contained in the subset of entities 402 may also be referred to as the third medical information. Likewise, the medical information contained in the subset of entities 404 may also be referred to as the fourth medical information. The semantic correlation may represent the correlation between the third and fourth medical information associated with the same target symptom of a disease or the same body part. That is, the semantic correlation represents the semantic concordance between the medical report 102 and the medical report 104 regarding the same target symptom of a disease or the same body part.

The similarity evaluation component 330 further determines the concordance level 440 based on the semantic correlation between the subsets of entities 402 and 404. For example, a higher semantic correlation may be mapped to a higher concordance level 440, for example a value close to and less than 1. A lower semantic correlation may be mapped to a lower concordance level 440, for example a value close to and greater than 0. It is to be understood that the values of 0 and 1 are only for the purpose of illustration, without suggesting any limitations. The value of concordance level 440 may be of any suitable range.

Several embodiments regarding determining the concordance level between different subsets of entities have been described with respect to FIG. 4. It is to be understood that the architecture 400 in FIG. 4 is only for the purpose of illustration. It can also use other suitable process to determine the concordance level between different subset of entities.

Similar to the concordance level 150, the concordance level 440 may indicate the correlation such as “match”, “partially match” or “mismatch” (also referred to as “no match”) between the subset of entities 402 and the subset of entities 404. The concordance level 440 may also be transmitted to the user interface 160. The user interface 160 may provide an indication based on the concordance level 440.

For example, the data processing system 110 may predetermine a third threshold level and a fourth threshold level larger than the third threshold level. If the concordance level 440 is below the third threshold level, the user interface 160 may provide an indication indicating that the third medical information mismatches with the fourth medical information. If the concordance level 440 exceeds the third threshold level and is below the fourth threshold level, the user interface 160 may provide an indication indicating that the third medical information partially matches with the fourth medical information. In addition, if the concordance level 440 exceeds the fourth threshold level, the user interface 160 may provide an indication indicating that the third medical information matches with the fourth medical information.

It is to be understood that the data processing system 110 may also predetermine only one threshold level, and the concordance level 440 may indicate that the third medical information matches or mismatches with the fourth medical information based on the concordance level 440.

By providing the indication by the user interface 160, it may help the users of the data processing system 110, such as doctors, radiologists and other healthcare professionals, to obtain comparison information between different medical reports. In this way, it may help the users to make diagnosis, and also facilitate peer learning and continuing education.

In some example embodiments, the user interface 160 may provide additional information based on the concordance level 440. For example, if the concordance level 440 is below the third threshold level, the user interface 160 may highlight, in the medical reports 102 and 104, at least one entity in the subset of entities 402 and the subset of entities 404. The at least one entity in the subsets of entities 402 and 404 indicates mismatched medical information. For example, if the subset of entities 402 comprise an entity representing “occupied lesion” associated with the left liver, while the subset of entities 404 comprise an entity representing “hemangiomas” associated with the left liver, then the user interface 160 may highlight the phrases “occupied lesion” and “hemangiomas”.

If the concordance level 440 exceeds the third threshold level and is below a fourth threshold level, the user interface 160 may highlight, in the medical reports 102 and 104, at least one entity in the subset of entities 402 and the subset of entities 404 indicating partially matched or mismatched medical information. Moreover, if the concordance level 440 exceeds the fourth threshold level, highlighting, in the first and second medical reports, the user interface 160 may highlight, in the medical reports 102 and 104, at least one entity in the subset of entities 402 and the subset of entities 404 indicating matched medical information.

By highlighting the words and phrases corresponding to the matched or mismatched entities, it may help the user to focus on the information with more importance. It will thus reduce the time of searching information from the reports and improve the efficiency and accuracy of the diagnosis result.

Example embodiments regarding providing indications and additional information by the user interface 160 have been described above. More details about the display of the user interface 160 may be described with respect to FIGS. 5-6 below.

FIG. 5 illustrates an example user interface 160 in accordance with some embodiments of the present disclosure. The user interface 160 presents overview of various types of medical reports associated with a patient. The user interface 160 may provide a variety of information, such as the total number of reports in different departments. For example, as shown in FIG. 5, there are 1,234 cardiology reports for the patient in year 2020. The user interface 160 also shows the average waiting time in year 2020. The pie charts in FIG. 5 illustrate proportions of reports by using different instruments, in different groups, in different departments and for patient with different genders.

FIG. 5 also illustrates several bar charts 510 which show the consistent rates of different reports. Those bar charts 510 illustrates the match rates, the partially match rates and the no match rates in different months in year 2020. The bar charts 510 also show that the average consistent rate is equal to 89%. The consistent rate is the sum of the match rate and the partially match rate.

By providing the above information via the user interface 160, it provides correlation based insights to users such as healthcare professionals. Particularly, the bar charts 510 which showing the consistent rate of different reports would help the researchers to notice the performance of different instruments providing those medical reports. This in turn will improve the development of those instruments.

FIG. 6 illustrates another example user interface 160 in accordance with some embodiments of the present disclosure. FIG. 6 shows radiology findings 610 and radiology diagnosis 615 of a selected radiology report. FIG. 6 also shows pathological diagnosis 620 of a selected pathological report. The selected radiology report and the selected pathology report are associated with a same liver tumor patient who undertook the lung CT and the pathology procedure in a hospital. The selected radiology report and the selected pathology report may be selected by using the method described with respect to FIG. 2.

FIG. 6 further illustrates several comparison results 630, 635, 640, 645 and 650 of the selected radiology report and the selected pathology report of the patient. The cross in the comparison result 630 indicates that the selected radiology and pathological reports mismatch with each other. The cross in the comparison result 630 indicates that the selected radiology and pathological reports mismatch with each other. The exclamation point in the comparison results 635 and 640 indicates that the selected radiology and pathological reports partially match with each other. By contrast, the tick in the comparison results 645 and 650 indicates that the selected radiology and pathological reports match with each other.

The user of the data processing system 110 may select a comparison result by clicking at the corresponding area in the user interface 160. For example, as illustrated in FIG. 6, the user selects the comparison result 645. Responsive to the selection, the user interface 160 will highlight the selected comparison result 645. In addition, the user interface 160 may further highlight the corresponding information in the radiology findings 610, the radiology diagnosis and the pathological diagnosis 620 associated with the comparison result 645.

In the example of FIG. 6, the comparison result 645 indicates that information from the two reports regarding the tumor of the right liver of the patient matches. Accordingly, words, phrases or sentence associated with the tumor of the right liver are underlined in the radiology findings 610, the radiology diagnosis and the pathological diagnosis 620 as shown in FIG. 6.

In this way, it may provide the underlined or highlighted text positions as correlation evidences. By providing correlated information between medical reports via the user interface, especially those highlighted text, it may provide insights for the healthcare professionals. Such insights would help the healthcare professionals to make diagnosis and facilitate peer learning.

It is to be understood that the above illustrated user interface 160 in FIGS. 5-6 are only for the purpose of illustration, without suggesting any limitation. Any suitable user interface can be applied. For example, in some example embodiments, the user interface 160 may also display the correlated reports and their correlations by different categories. By providing the correlation results in different categories, it may provide a care quality control tool for healthcare professionals such as radiology department heads.

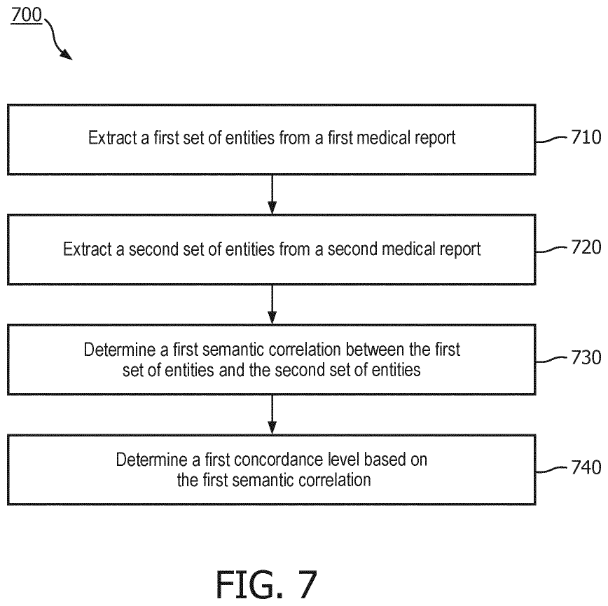

FIG. 7 illustrates a flow chart of a method 700 according to embodiments of the present disclosure. The method 700 may be implemented by the data processing system 110. It is to be understood that the method 700 may also be implemented by any other suitable device or apparatus.

As shown in FIG. 7, at block 710, the data processing system 110 extracts a first set of entities from a first medical report. The first medical report presents first medical information related to a patient. At block 720, the data processing system 110 extracts a second set of entities from a second medical report. The second medical report presents second medical information related to the patient. At block 730, the data processing system 110 determines a first semantic correlation between the first set of entities and the second set of entities. At block 740, the data processing system 110 determines, based on the first semantic correlation, a first concordance level between the first medical information in the first medical report and the second medical information in the second medical report.

In some example embodiments, the method 700 further comprises: in accordance with a determination that the first concordance level is below a first threshold level, providing a first indication indicating that the first medical information mismatches with the second medical information; in accordance with a determination that the first concordance level exceeds the first threshold level and is below a second threshold level, providing a second indication indicating that the first medical information partially matches with the second medical information, the second threshold level being larger than the first threshold level; and in accordance with a determination that the first concordance level exceeds the second threshold level, providing a third indication indicating that the first medical information matches with the second medical information.

In some example embodiments, the method 700 further comprises: associating each entity of the first set of entities and the second set of entities with a respective symptom of a disease or a respective body part; determining a first subset of entities from the first set of entities and a second subset of entities from the second set of entities, the first subset of entities and the second subset of entities being associated with a same target symptom of a disease or a same target body part; determining a second semantic correlation between the first subset of entities and the second subset of entities; and determining, based on the second semantic correlation, a second concordance level between third medical information associated with the target symptom of a disease or the target body part in the first medical report and fourth medical information associated with the target symptom of a disease or the same body part in the second medical report.

In some example embodiments, associating each entity of the first set of entities and the second set of entities with a respective symptom of a disease or a respective body part comprises: associating, based on a knowledge base, each entity of the first set of entities and the second set of entities with a respective symptom of a disease or a respective body part, the knowledge base comprising a plurality of medical concepts and relations.

In some example embodiments, the method 700 further comprises: in accordance with a determination that the second concordance level is below a third threshold level, providing a fourth indication indicating that the third medical information mismatches with the fourth medical information; in accordance with a determination that the second concordance level exceeds the third threshold level and is below a fourth threshold level, providing a fifth indication indicating that the third medical information partially matches with the fourth medical information, the fourth threshold level being larger than the third threshold level; and in accordance with a determination that the second concordance level exceeds the fourth threshold level, providing a sixth indication indicating that the third medical information matches with the fourth medical information.

In some example embodiments, the method 700 further comprises: in accordance with a determination that the second concordance level is below a third threshold level, highlighting, in the first and second medical reports, at least one entity in the first subset of entities and the second subset of entities that indicate mismatched medical information; in accordance with a determination that the second concordance level exceeds the third threshold level and is below a fourth threshold level, highlighting, in the first and second medical reports, at least one entity in the first subset of entities and the second subset of entities that indicates partially matched or mismatched medical information, the fourth threshold level being larger than the third threshold level; and in accordance with a determination that the second concordance level exceeds the fourth threshold level, highlighting, in the first and second medical reports, at least one entity in the first subset of entities and the second subset of entities that indicates matched medical information.

In some example embodiments, the method 700 further comprises: obtaining a plurality of medical reports of the patient; determining text similarities between the plurality of medical reports of the patient; and selecting, from the plurality of medical reports and based on the text similarities, the first medical report and the second medical report, the first medical report and the second medical report being of different report types.

In some example embodiments, at least one of the first and second medical reports comprises at least one of the following: a radiology report, a pathology report, a medical laboratory report, an ultrasound report, or a discharge summary report.

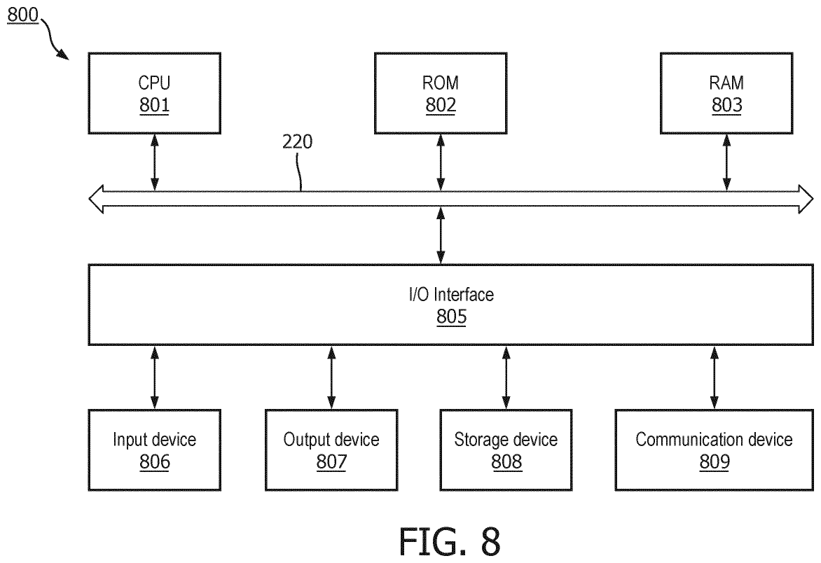

FIG. 8 illustrates a schematic diagram of an example device 800 for implementing embodiments of the present disclosure. For example, the data processing system 110 as shown in FIG. 1 can be implemented by the device 800. As shown, the device 800 includes a central process unit (CPU) 801, which can execute various suitable actions and processing based on the computer program instructions stored in the read-only memory (ROM) 802 or computer program instructions loaded in the random-access memory (RAM) 803 from a storage unit 808. The RAM 803 can also store all kinds of programs and data required by the operation of the device 800. CPU 801, ROM 802 and RAM 803 are connected to each other via a bus 804. The input/output (I/O) interface 805 is also connected to the bus 804.

A plurality of components in the device 800 is connected to the I/O interface 805, including: an input unit 806, such as keyboard, mouse and the like; an output unit 807, e.g., various kinds of display and loudspeakers etc.; a storage unit 808, such as disk and optical disk etc.; and a communication unit 809, such as network card, modem, wireless transceiver and the like. The communication unit 809 allows the device 800 to exchange information/data with other devices via the computer network, such as Internet, and/or various telecommunication networks.

The above described each procedure and processing, such as method 700, can also be executed by the processing unit 801. For example, in some embodiments, the method 700 can be implemented as a computer software program tangibly included in the machine-readable medium, e.g., storage unit 808. In some embodiments, the computer program can be partially or fully loaded and/or mounted to the device 800 via ROM 802 and/or communication unit 809. When the computer program is loaded to RAM 803 and executed by the CPU 801, one or more actions of the above described method 500 can be implemented.

The present disclosure can be method, apparatus, system and/or computer program product. The computer program product can include a computer-readable storage medium, on which the computer-readable program instructions for executing various aspects of the present disclosure are loaded.

The computer-readable storage medium can be a tangible apparatus that maintains and stores instructions utilized by the instruction executing apparatuses. The computer-readable storage medium can be, but not limited to, such as electrical storage device, magnetic storage device, optical storage device, electromagnetic storage device, semiconductor storage device or any appropriate combinations of the above. More concrete examples of the computer-readable storage medium (non-exhaustive list) include: portable computer disk, hard disk, random-access memory (RAM), read-only memory (ROM), erasable programmable read-only memory (EPROM or flash), static random-access memory (SRAM), portable compact disk read-only memory (CD-ROM), digital versatile disk (DVD), memory stick, floppy disk, mechanical coding devices, punched card stored with instructions thereon, or a projection in a slot, and any appropriate combinations of the above. The computer-readable storage medium utilized here is not interpreted as transient signals per se, such as radio waves or freely propagated electromagnetic waves, electromagnetic waves propagated via waveguide or other transmission media (such as optical pulses via fiber-optic cables), or electric signals propagated via electric wires.

The described computer-readable program instruction can be downloaded from the computer-readable storage medium to each computing/processing device, or to an external computer or external storage via Internet, local area network, wide area network and/or wireless network. The network can include copper-transmitted cable, optical fiber transmission, wireless transmission, router, firewall, switch, network gate computer and/or edge server. The network adapter card or network interface in each computing/processing device receives computer-readable program instructions from the network and forwards the computer-readable program instructions for storage in the computer-readable storage medium of each computing/processing device.

The computer program instructions for executing operations of the present disclosure can be assembly instructions, instructions of instruction set architecture (ISA), machine instructions, machine-related instructions, microcodes, firmware instructions, state setting data, or source codes or target codes written in any combinations of one or more programming languages, wherein the programming languages consist of object-oriented programming languages, e.g., Smalltalk, C++ and so on, and traditional procedural programming languages, such as “C” language or similar programming languages. The computer-readable program instructions can be implemented fully on the user computer, partially on the user computer, as an independent software package, partially on the user computer and partially on the remote computer, or completely on the remote computer or server. In the case where remote computer is involved, the remote computer can be connected to the user computer via any type of networks, including local area network (LAN) and wide area network (WAN), or to the external computer (e.g., connected via Internet using the Internet service provider). In some embodiments, state information of the computer-readable program instructions is used to customize an electronic circuit, e.g., programmable logic circuit, field programmable gate array (FPGA) or programmable logic array (PLA). The electronic circuit can execute computer-readable program instructions to implement various aspects of the present disclosure.

Various aspects of the present disclosure are described here with reference to flow chart and/or block diagram of method, apparatus (device) and computer program products according to embodiments of the present disclosure. It should be understood that each block of the flow chart and/or block diagram and the combination of various blocks in the flow chart and/or block diagram can be implemented by computer-readable program instructions.

The computer-readable program instructions can be provided to the processor of general-purpose computer, dedicated computer or other programmable data processing apparatuses to manufacture a machine, such that the instructions that, when executed by the processing unit of the computer or other programmable data processing apparatuses, generate an apparatus for implementing functions/actions stipulated in one or more blocks in the flow chart and/or block diagram. The computer-readable program instructions can also be stored in the computer-readable storage medium and cause the computer, programmable data processing apparatus and/or other devices to work in a particular manner, such that the computer-readable medium stored with instructions contains an article of manufacture, including instructions for implementing various aspects of the functions/actions stipulated in one or more blocks of the flow chart and/or block diagram.

The computer-readable program instructions can also be loaded into computer, other programmable data processing apparatuses or other devices, so as to execute a series of operation steps on the computer, other programmable data processing apparatuses or other devices to generate a computer-implemented procedure. Therefore, the instructions executed on the computer, other programmable data processing apparatuses or other devices implement functions/actions stipulated in one or more blocks of the flow chart and/or block diagram.

The flow chart and block diagram in the drawings illustrate system architecture, functions and operations that may be implemented by system, method and computer program product according to multiple implementations of the present disclosure. In this regard, each block in the flow chart or block diagram can represent a module, a part of program segment or code, wherein the module and the part of program segment or code include one or more executable instructions for performing stipulated logic functions. In some alternative implementations, it should be noted that the functions indicated in the block can also take place in an order different from the one indicated in the drawings. For example, two successive blocks can be in fact executed in parallel or sometimes in a reverse order dependent on the involved functions. It should also be noted that each block in the block diagram and/or flow chart and combinations of the blocks in the block diagram and/or flow chart can be implemented by a hardware-based system exclusive for executing stipulated functions or actions, or by a combination of dedicated hardware and computer instructions.

Various embodiments of the present disclosure have been described above and the above description is only exemplary rather than exhaustive and is not limited to the embodiments of the present disclosure. Many modifications and alterations, without deviating from the scope and spirit of the explained various embodiments, are obvious for those skilled in the art. The selection of terms in the text aims to best explain principles and actual applications of each embodiment and technical improvements made in the market by each embodiment, or enable those ordinary skilled in the art to understand embodiments of the present disclosure.

1. A method for data processing, comprising:
extracting a first set of entities from a first medical report, the first medical report presenting first medical information related to a patient;
extracting a second set of entities from a second medical report, the second medical report presenting second medical information related to the patient;
determining a first semantic correlation between the first set of entities and the second set of entities; and
determining, based on the first semantic correlation, a first concordance level between the first medical information in the first medical report and the second medical information in the second medical report.

2. The method of claim 1, further comprising:
in accordance with a determination that the first concordance level is below a first threshold level, providing a first indication indicating that the first medical information mismatches with the second medical information;
in accordance with a determination that the first concordance level exceeds the first threshold level and is below a second threshold level, providing a second indication indicating that the first medical information partially matches with the second medical information, the second threshold level being larger than the first threshold level; and
in accordance with a determination that the first concordance level exceeds the second threshold level, providing a third indication indicating that the first medical information matches with the second medical information.

3. The method of claim 1, further comprising:
associating each entity of the first set of entities and the second set of entities with a respective symptom of a disease or a respective body part;
determining a first subset of entities from the first set of entities and a second subset of entities from the second set of entities, the first subset of entities and the second subset of entities being associated with a same target symptom of a disease or a same target body part;
determining a second semantic correlation between the first subset of entities and the second subset of entities; and
determining, based on the second semantic correlation, a second concordance level between third medical information associated with the target symptom of a disease or the target body part in the first medical report and fourth medical information associated with the target symptom of a disease or the same body part in the second medical report.

4. The method of claim 3, wherein associating each entity of the first set of entities and the second set of entities with a respective symptom of a disease or a respective body part comprises:
associating, based on a knowledge base, each entity of the first set of entities and the second set of entities with a respective symptom of a disease or a respective body part, the knowledge base comprising a plurality of medical concepts and relations.

5. The method of claim 3, further comprising:
in accordance with a determination that the second concordance level is below a third threshold level, providing a fourth indication indicating that the third medical information mismatches with the fourth medical information;
in accordance with a determination that the second concordance level exceeds the third threshold level and is below a fourth threshold level, providing a fifth indication indicating that the third medical information partially matches with the fourth medical information, the fourth threshold level being larger than the third threshold level; and
in accordance with a determination that the second concordance level exceeds the fourth threshold level, providing a sixth indication indicating that the third medical information matches with the fourth medical information.

6. The method of claim 3, further comprising:
in accordance with a determination that the second concordance level is below a third threshold level, highlighting, in the first and second medical reports, at least one entity in the first subset of entities and the second subset of entities that indicate mismatched medical information;
in accordance with a determination that the second concordance level exceeds the third threshold level and is below a fourth threshold level, highlighting, in the first and second medical reports, at least one entity in the first subset of entities and the second subset of entities that indicates partially matched or mismatched medical information, the fourth threshold level being larger than the third threshold level; and
in accordance with a determination that the second concordance level exceeds the fourth threshold level, highlighting, in the first and second medical reports, at least one entity in the first subset of entities and the second subset of entities that indicates matched medical information.

7. The method of claim 1, further comprising:
obtaining a plurality of medical reports of the patient;
determining text similarities between the plurality of medical reports of the patient; and
selecting, from the plurality of medical reports and based on the text similarities, the first medical report and the second medical report, the first medical report and the second medical report being of different report types.

8. The method of claim 1, wherein at least one of the first and second medical reports comprises at least one of the following:
a radiology report,
a pathology report,
a medical laboratory report,
an ultrasound report, or
a discharge summary report.

9. A computing system comprising:
at least one processor; and
at least one memory comprising computer readable instructions which, when executed by the at least one processor of an electronic device, cause the electronic device to perform operations comprising:
extracting a first set of entities from a first medical report, the first medical report presenting first medical information related to a patient;
extracting a second set of entities from a second medical report, the second medical report presenting second medical information related to the patient;
determining a first semantic correlation between the first set of entities and the second set of entities; and
determining, based on the first semantic correlation, a first concordance level between the first medical information in the first medical report and the second medical information in the second medical report.

10. The computing system of claim 9, the operations further comprising:
in accordance with a determination that the first concordance level is below a first threshold level, providing a first indication indicating that the first medical information mismatches with the second medical information;
in accordance with a determination that the first concordance level exceeds the first threshold level and is below a second threshold level, providing a second indication indicating that the first medical information partially matches with the second medical information, the second threshold level being larger than the first threshold level; and
in accordance with a determination that the first concordance level exceeds the second threshold level, providing a third indication indicating that the first medical information matches with the second medical information.

11. The computing system of claim 9, the operations further comprising:
associating each entity of the first set of entities and the second set of entities with a respective symptom of a disease or a respective body part;
determining a first subset of entities from the first set of entities and a second subset of entities from the second set of entities, the first subset of entities and the second subset of entities being associated with a same target symptom of a disease or a same target body part;
determining a second semantic correlation between the first subset of entities and the second subset of entities; and
determining, based on the second semantic correlation, a second concordance level between third medical information associated with the target symptom of a disease or the target body part in the first medical report and fourth medical information associated with the target symptom of a disease or the same body part in the second medical report.

12. The computing system of claim 11, wherein associating each entity of the first set of entities and the second set of entities with a respective symptom of a disease or a respective body part comprises:
associating, based on a knowledge base, each entity of the first set of entities and the second set of entities with a respective symptom of a disease or a respective body part, the knowledge base comprising a plurality of medical concepts and relations.

13. The computing system of claim 11, the operations further comprising:
in accordance with a determination that the second concordance level is less than a third threshold level,
providing a fourth indication indicating that the third medical information mismatches with the fourth medical information; and
highlighting, in the first and second medical reports, at least one entity in the first subset of entities and the second subset of entities that indicate mismatched medical information;
in accordance with a determination that the second concordance level exceeds the third threshold level and is below a fourth threshold level, the fourth threshold level being larger than the third threshold level,
providing a fifth indication indicating that the third medical information partially matches with the fourth medical information; and
highlighting, in the first and second medical reports, at least one entity in the first subset of entities and the second subset of entities that indicate partially matched or mismatched medical information; and
in accordance with a determination that the second concordance level exceeds the fourth threshold level,
providing a sixth indication indicating that the third medical information matches with the fourth medical information; and
highlighting, in the first and second medical reports, at least one entity in the first subset of entities and the second subset of entities that indicates matched medical information.

14. The computing system of claim 9, the operations further comprising:
obtaining a plurality of medical reports of the patient;
determining text similarities between the plurality of medical reports of the patient; and
selecting, from the plurality of medical reports and based on the text similarities, the first medical report and the second medical report, the first medical report and the second medical report being of different report types.

15. A computer readable medium comprising instructions which, when executed by at least one processor, cause the at least one processor to perform the method according to claim 1.
